(19) United States
(12) Patent Application Publication
(10) Pub. No.: US 2011/0319805 A1
(43) Pub. Date: Dec. 29, 2011

(54) Topical composition for treating the skin

(76) Inventor: Bryan Knicely Morris, Charlotte Courthouse, VA (US)

(21) Appl. No.: 13/067,683

(22) Filed: Jun. 21, 2011

Related U.S. Application Data
(60) Provisional application No. 61/344,321, filed on Jun. 29, 2010.

(51) Int. Cl. A61K 31/79; A61L 15/00; A61P 17/00; A61P 31/04; A61P 31/10

(52) U.S. Cl. 602/48; 424/78.06

(57) ABSTRACT

The present invention relates to a topical composition for the treatment of mammals suffering from a condition of the skin such as a skin lesion, a wound, a cut, an abrasion, a hot spot, a sore foot pad, inflamed skin or dry skin; wherein the composition contains a blend of four separate creams, a steroidal material, a normally injectable vitamin A and vitamin D composition, an antibiotic material and, optionally, ingredients such as aloe vera, an antifungal agent, an anti-parasitic agent, DMSO and a topical anaesthetic.

The present application claims the benefit of U.S. Provisional Application No. 61/344,321, filed on Jun. 29, 2010.

### FIELD OF THE INVENTION

The present invention relates to a topical composition that has utility in the field of medicinal and veterinary treatment of mammals that are suffering from a condition of the skin. The condition of the skin can be a skin lesion, a wound, a cut or an abrasion. Further, the condition can be a hot spot, a sore foot pad of an animal such as a dog, inflamed skin, infected skin or even dry skin.

### BACKGROUND OF THE INVENTION

Compositions such as gels, ointments, salves and the like are useful in treating conditions of the skin. Compositions that readily coat the skin and at the same time provide effective adhesion to the skin surface are preferred. Further, it is useful to have a composition that provides soothing relief to the skin as well as healing activity.

In the past, various formulations were employed to treat the skin of mammals. A review of some of the prior art formulations is hereby included below.

US Patent Application Publication No. 2009/0317354 relates to a topical composition for repairing injured skin. The composition contains 50 to 90% by weight of saccharide, 0.5 to 10% by weight of povidone-iodine, 0.1 to 20% by weight of water and 0.3 to 5% by weight of phospholipid.

US Patent Application Publication No. 2009/0304812 relates to a pharmaceutical composition for topical application to a mammalian patient. The composition contains a therapeutic agent and a pharmaceutically acceptable carrier. The composition is solid at a temperature of about 25 degrees C. or less, and, upon continuous contact with the skin of the patient, softens to a consistency to effect substantial application of the therapeutic agent to a desired skin area of the mammalian patient within a time period of less than 10 minutes. The therapeutic agent is a keratolytic agent, hormone modifier, steroid, antihistaminic, antibiotic, anti-fungal agent, anti-infection agent, antimicrobial agent, anti-viral agent, antiseptic, immunosuppressant agent, or an anti-ageing agent. Examples of therapeutic agents are benzoyl peroxide, zinc, a vitamin D analogue, 5-fluoro uracil, salicylic acid, formaldehyde, glutaraldehyde, silver nitrate, imiquimod or podophyllum.

US Patent Application Publication No. 2009/0297616 discloses a method for accelerating healing of wounds resulting from cutaneous surgery. The composition contains petrolatum ointment and zinc oxide. The zinc oxide is present in the composition in an amount of about 1% to about 10% by weight. The wound care composition can further contain one or more ingredients such as antimicrobial ingredients, preservatives, ingredients for promotion of cell growth, and moisture barriers. The composition further includes one or more ingredients such as silver, hyaluronic acid and silicone.

US Patent Application Publication No. 2009/0162446 relates to a process for preparing a skin care composition and applying the skin care composition to an absorbent article. The process includes providing a carrier; mixing a pre-dispersant mixture comprising a skin care ingredient and a dispersant fluid; combining the pre-dispersant mixture with the carrier to form the skin care composition; and applying the skin care composition to at least a portion of the absorbent article. The carrier can be an emollient such as petrolatum. The skin care ingredient can be zinc oxide, talc, starch, allantoin, hexamidine, salts of hexamidine, derivatives of hexamidine, triacetin, phytic acid, ethylenediamine tetraacetic acid (EDTA), 4-(2-aminoethyl)-benzenesulfonylfluoride hydrochloride and chitosan.

US Patent Application Publication No. 2009/0041859 discloses a wound treatment drug for external use. The drug contains an oily base and iodine. The drug does not contain white soft sugar. The oily base can be petrolatum. The amount of iodine is 0.01% to 5% by weight. The drug can be in the form of a paste, ointment, cream, liquid, gel, adhesive, cataplasm or patch.

US Patent Application Publication No. 2008/0057138 discloses a formulation for topical application to the skin of a human being. The formulation contains a first component and a second component. The first component includes vitamin C in a concentration of about 5% to 30% by weight of the formulation; vitamin E in a concentration of about 1% to about 10% by weight of the formulation; thickeners, emulsifiers, emollients and moisturizers. The second component includes vitamin A in a concentration of about 0.2% to about 10% by weight of the formulation (or retinol in a concentration of about 0.2% to about 10% by weight of the composition, or both vitamin A and retinol); vitamin E in a concentration of about 1% to about 10% by weight of the formulation; zinc sulphate in a concentration of about 2% to about 5% by weight of the formulation; and skin compatible thickeners.

US Patent Application Publication No. 2008/0008759 discloses a method of enhancing the natural healing of a wound. The method includes applying an uncured ionomer-resin to the surface of the wounded area. The uncured ionomer-resin can contain fluoride, healing agents, antibiotic agents and antimicrobial agents. The antimicrobial agent can be isoniazid, ethambutol, clofazimine, rifabutin, fluoroquinolones, pyrazinamide, streptomycin, ofloxacin, ganciclovir, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, erythromycin, clindamycin, lincomycin, acyclovir, trifluorouridine, pentamidine, atovaquone, paromomycin, diclazaril, acyclovir, trifluorouridine, foscamet, penicillin, gentamicin or sparfloxacin.

US Patent Application Publication No. 2007/0275093 relates to a method for treating a non-microbial inflammatory skin condition. The method includes administering a topical formulation containing imidazole or a salt thereof, and a carrier. The imidazole can be miconazole, ketoconazole, econazole and isoconazole.

US Patent Application Publication No. 2004/0062817 discloses a therapeutic tissue healing composition containing an effective amount of a mixture of fat-soluble vitamins A and D.

US Patent Application No. 2003/0194446 discloses a topical therapeutic composition containing 20% zinc oxide; 0.025-5% of a therapeutically active agent, wherein the active agent can be adrenocortical steroids, Polymyxin B Sulfate, Bacitracin Zinc and Neomycin, Quinoline, Betamethasone dipropionate, Pramoxime hydrochloride, Clotrimazole, Ketoconazole; and a base. The base is a mixture of at least two different compounds. The compounds include petrolatum, wax, mineral oil and lanolin.

U.S. Pat. No. 7,115,287 discloses a topical medicament for treating a dermatologic condition. The medicament contains vitamin A oil, vitamin D oil, vitamin E oil, lanolin, petroleum jelly, colloidal silver, ti tree oil, zinc oxide and corn starch.

U.S. Pat. No. 7,094,431 discloses a therapeutic tissue healing composition containing zinc oxide, fat-soluble vitamins A, D, E, K, an antibacterial agent, an antifungal agent, and a calcium channel blocker.

U.S. Pat. No. 6,660,306 discloses a method of healing skin wounds in mammals. The method includes topically contacting a mammalian skin wound with a therapeutic composition. The composition contains zinc oxide in an amount of about 2% to about 25% by weight; vitamin A, vitamin D, vitamin E, and vitamin K, wherein the four vitamins are present in the therapeutic composition in a combined amount of 1 to 35 wt % bt weight; bacitracin zinc and clotrimazole.

U.S. Pat. No. 6,570,054 discloses a skin care composition which is disposed on at least a portion of an article. The skin care composition contains a carrier; a skin care ingredient; and an effective amount of a rheological agent. The composition is at least partially transferable to skin when the article is placed next to the skin.

U.S. Pat. No. 6,419,936 discloses a topical skin formulation. The formulation contains vitamin E in an oil base; vitamin A ointment; vitamin D ointment; zinc oxide ointment; and aloe vera extract.

U.S. Pat. No. 6,375,942 discloses an ointment for the treatment of human skin. The ointment includes wax (20.0% by weight); petrolatum (74.45% by weight); antibiotic (1.05% by weight); zinc oxide (4.0% by weight); and an anti-itch ingredient (0.5% by weight).

U.S. Pat. No. 6,261,574 discloses a cream formulation for topical application to an external portion of a human body. A one ounce preparation of the cream formulation includes an aloe vera gel compound in an amount of about 3 grams to about 10 grams; a zinc compound in an amount of about 0.4 grams to about 1.5 grams; vitamin E in an amount of about 200 IU to about 9000 IU; and the balance being water, organic solvents, carriers and emollients.

U.S. Pat. No. 6,251,423 relates to a paste formulation for topical application to a body. The formulation has a creamy consistency. The formulation contains an emulsion of a wax or oil in an aqueous gel of a water-insoluble polymeric material together with at least one emulsifier and an active medicinal ingredient.

U.S. Pat. No. 5,905,092 discloses a composition for the treatment of wounds. The composition contains a topical semisolid, an antibiotic formulation and at least 60% by weight of water. The topical semisolid contains from about 10% to about 20% by weight of a polyhydric alcohol and from about 0.5% to about 10% by weight each of two or more gelling agents.

U.S. Pat. No. 5,874,094 discloses a cream formulation for topical application to an external portion of a human body. A one ounce preparation of the cream formulation includes an aloe vera gel compound in a weight range of between about 3 grams and about 10 grams; a zinc compound in a weight range of between about 0.4 gram and about 1.5 grams; vitamin E in a weight range of between about 200 IU and about 9000 IU; an extract from Eriobotrya japonica in a weight range of between about 3 mg and about 15mg; and the balance being made up of water, organic solvents, carriers and emollients.

U.S. Pat. No. 5,631,301 discloses a composition for the treatment of wounds. The composition contains a topical semisolid composition and an antibiotic formulation, wherein the topical semisolid contains a hydrophilic gel or hydroalcoholic gel having between about 1% to about 4% ammonium acrylates/acrylonitrogen copolymer as the gelling agent.

U.S. Pat. No. 4,671,957 discloses an antibacterial cream for topical application. The cream contains polyvinylpyrrolidone-iodine distributed in an oil-in-water emulsion, water, and a mixture of at least one hydrocarbon component and at least one polyol moisturizing component. The hydrocarbon component can be petrolatum.

None of the above-cited references, taken either alone or in combination, anticipates the present invention.

### SUMMARY OF THE INVENTION

A topical composition of the present invention includes a blend of four separate creams. A. first cream contains vitamin A and vitamin D. A second cream contains povidone-iodine. A third cream contains ichthammol. A fourth cream contains “thuja zinc” (a mixture of oil of thuja and zinc oxide) and scarlet oil. The topical composition further contains at least one steroid. The topical composition also contains an injectable composition containing a mixture of vitamin A, vitamin D and a liquid carrier. The injectable composition is not employed in its usual manner, but rather mixed into the topical composition. Another ingredient in the topical composition is an antibiotic material such as tetracycline or the like. Optionally, the topical composition can include such ingredients as aloe vera, a fungicide, a topical anaesthetic, a skin softener, an anti-parasitic agent and dimethylsulfoxide.

### DETAILED DESCRIPTION OF THE INVENTION

Topical compositions of the present invention are useful in the field of veterinary medicine. These compositions can also be employed in various practices relating to human patients, such as in dermatology and wound care management.

In an embodiment, the topical composition contains a blend of four separate creams. A first cream contains vitamin A and vitamin D. The vitamins are mixed with a base. Preferably, the base is petrolatum or lanolin. A second cream contains povidone-iodine, a complex of the polymer polyvinylpyrrolidone (PVP) and iodine. An emollient base is also present. A third cream contains ichthammol and an emollient base. A fourth cream contains oil of thuja, zinc oxide, scarlet oil and petrolatum as a base. Preferably, the blend of the four separate topical creams is made up of about four pounds of the first cream, about one pound of the second cream, about one pound of the third cream and about one pound of the fourth cream. It is within the scope of the present invention to employ fractions of the above-disclosed weights, or multiples of the above-disclosed weights; provided the ratios of the four topical creams remain constant.

The topical composition further contains a steroidal material. By steroidal material is meant a composition that contains at least one steroid and a base such as white petrolatum. Steroids useful in the present invention are: hydrocortisone, betamethasone, dexamethasone, flumethasone, isoflupredone, methylprednisolone, prednisone, prednisolone, triamcinalone, fludricortisone, paramethasone and clobetasol. Pharmaceutically active derivatives of each of the above-listed steroids, such as 6-alpha-methylprednisolone, can also be employed. Preferably, the steroidal material is present in the topical composition in an amount of about one pound. It is within the scope of the present invention to employ fractions of the above-disclosed weight, or multiples of the above-disclosed weight; provided the ratios of the steroidal material and the four topical creams remain constant.

The topical composition further contains an injectable composition containing a mixture of vitamin A and vitamin D. The injectable composition can further contain vitamin E. The composition further contains a liquid carrier such as water. By injectable composition is meant a free flowing material that is readily administered to a patient through the skin in an intravenous or subcutaneous manner by means of a syringe or the like. Such injectable compositions are known in the art. In one embodiment, the injectable composition is present in the topical composition in an amount of about 30 ml to about 50 ml. It is within the scope of the present invention to employ fractions of the above-disclosed volumes, or multiples of the above-disclosed volumes; provided the ratios of the injectable composition, the steroidal material and the four topical creams remain constant.

Another part of the topical composition is an antibiotic material. By antibiotic material is meant a composition that contains at least one antibiotic. Classes of antibiotics that can be employed in the present invention are: cephalosporines, floroquinolines, aminoglycosides, sulfas, macrolides, and tetracyclines. Specific examples of antibiotics are: cephalexin, cephadrine, cefaclor, cefuroxime, cefodroxil, cefpodoxime, cefixime, orbifloxacin, enrofloxacin, ciprofloxacin, difloxacin, marbofloxacin, danofloxacin, amikacin, kanamycin, gentamycin, neomycin, polymyxin, bacitracin, trimethoprim sulfa, sulfadimethoxine, azithromycin, erythromycin, clarithromycin, clindamycin, tetracycline, oxytetracycline, chlortetracycline, doxycycline, florfenicol and spectinomycin. Pharmaceutically active derivatives of each of the above-listed antibiotics can also be employed. Preferably, the antibiotic material is present in the topical composition in an amount of about 30 ml to about 50 ml. It is within the scope of the present invention to employ fractions of the above-disclosed volumes, or multiples of the above-disclosed volumes; provided the ratios of the antibiotic material, the injectable composition, the steroidal material and the four topical creams remain constant.

Additional ingredients that are used in the topical composition on an optional basis include aloe vera, an antifungal agent, a topical anaesthetic, a skin softener, an anti-parasitic agent, and dimethylsulfoxide (DMSO). Classes of antifungal agents are: imidazoles and triazoles. An antifungal agent from any of these classes can be employed as an ingredient in the topical composition of the present invention. Specific examples of antifungal agents are miconazole and ketoconazole. Specific examples of anti-parasitic agents are: amitraz, rotenone and ivermectin. Anti-parasitic agents are useful in the treatment of a specific skin disease called demodectic mange caused by a normally occurring skin mite known as demodex. Mixtures of optional ingredients can be employed, such as a mixture of aloe vera and miconazole. Preferably, the optional ingredient or the mixture of optional ingredients is present in the topical composition in an amount of about 30 ml to about 50 ml. It is within the scope of the present invention to employ fractions of the above disclosed volumes, or multiples of the above-disclosed volumes; provided the ratios of the optional ingredient(s), the antibiotic material, the injectable composition, the steroidal material and the four topical creams remain constant.

The topical composition of the present invention is applied directly to the site of the skin condition as by spreading by hand or by use of an applicator. Preferably, the entire site of the lesion, wound, cut, abrasion or the like is completely covered by the application of the topical composition. It is within the scope of the present invention to employ two or more coatings of the composition to cover the relevant area of the skin. Alternatively, the topical composition of the present invention is a part of a bandage or the like. The bandage is pre-treated with the topical composition of the present invention. One method of pre-treatment of the bandage is by an impregnation process. The bandage, containing the topical composition, is then applied directly over the area of insult on the skin.

The topical composition is applied to the site of the skin condition by means of an applicator, especially when DMSO is one of the ingredients in the composition of the present invention. An applicator which is coated with or contains the topical composition of the present invention is employed. In one embodiment, the applicator can be a hand-held device that includes a roller, a roller mechanism and a hand-grip. The roller can contain the topical composition on the surface of the roller or embedded within the roller. Various means are employed to retain the topical composition on the roller. Such means include absorption, adsorption, coating, containment or the like. In an alternative embodiment, the applicator contains a chamber for storing and releasing the topical composition. Preferably, the chamber includes flexible walls wherein at least one of the flexible walls contains a plurality of apertures for release of the topical composition onto the skin. In one embodiment, the chamber can be a part of the hand-held device containing the roller.

The present invention relates to a method for the preparation of a topical composition that is useful in the treatment of mammals that are suffering from a condition of the skin such as a skin lesion, a wound or the like. A mixing device is employed for the blending of the various ingredients. The mixing device contains a heating unit so that the various ingredients can be heated both prior to and during the mixing operation. Heating of the ingredients assists in the formation of a homogeneous mixture of components. In an embodiment, the mixing device is a Waring blender.

The method of the present invention includes providing a mixing device; heating the mixing device to a temperature of about 30 degrees C. to about 50 degrees C.; adding to the heated mixing device a first cream containing vitamin A, vitamin D and a base, wherein the base is petrolatum, lanolin or mixtures thereof; and adding to the heated mixing device a second cream containing polyvinylpyrrolidone-iodine and an emollient base. The mixing device is optionally activated to form a blend of the first cream and the second cream as by a mixing operation. The method further includes adding to the heated mixing device a third cream containing ichthammol and an emollient base; optionally, activating the mixing device to form a blend of the first second, and third creams; adding to the heated mixing device a fourth cream containing oil of thuja, zinc oxide, scarlet oil and petrolatum as a base; and, optionally, activating the mixing device to form a blend of the first, second, third and fourth creams. In an alternative embodiment, the first, second, third and fourth creams are added to the heated mixing device in any order. In yet another alternative embodiment, but less preferred, the first, second, third and fourth creams are added all at once to the heated mixing device. The method further includes adding to the heated mixing device a steroidal material containing at least one steroid; optionally, activating the mixing device to form a blend of four creams (the first, second, third and fourth creams) and the steroidal material; adding to the heated mixing device an injectable composition containing vitamin A, vitamin D and a liquid carrier; optionally, activating the mixing device to form a blend of the four creams, the steroidal material, and the injectable composition as by a mixing operation; adding to the heated mixing device an antibiotic material containing at least one antibiotic; and, optionally, activating the mixing device to form a blend of the four creams, the steroidal material, the injectable composition and the antibiotic material. The method further includes adding an optional ingredient or mixture of optional ingredients to the heated mixing device; and activating the mixing device to form a blend of the four creams, the steroidal material, the injectable composition, the antibiotic material and the optional ingredient (if present). The topical composition is then withdrawn from the mixing device. Preferably, the optional ingredient is aloe vera, an antifungal agent such as ketoconazole, a topical anaesthetic such as lidocaine, DMSO or an anti-parasitic agent. Mixtures of optional ingredients can also be employed, such as a blend of aloe vera and lidocaine. The topical composition that is withdrawn from the mixing device has a creamy smooth texture and is homogeneous in character.

With regard to the method step of heating the mixing device, the heating can be performed either by a self-contained heating element built into the mixing device or by a separate heating unit positioned adjacent to the mixing device.

The present invention also relates to a method for the treatment of mammals suffering from a condition of the skin. The condition of the skin is a skin lesion, a wound, a cut, an abrasion, a hot spot, a sore foot pad, inflamed skin, infected skin or dry skin. It is also within the scope of the present invention to treat calloused, sunburned or weathered skin. It is within the scope of the present invention to treat minor burns, rashes, poison ivy or the like. Other conditions of the skin that are treatable with the composition of the present invention are: rashes, boils, insect bites, allergic reactions, dermatitis, itchiness, bruises and scabies. It is further within the scope of the present invention to employ the composition described herein in conjunction with other compositions for treating the skin, for example, pharmaceutical compositions for use in dermatology or for use in the treatment of skin cancer. In general, any condition of mammalian skin can be treated with the topical composition. Alternatively, the inventive composition can be used to supplement or enhance other treatments.

In an embodiment, the method for the treatment of the skin of mammals, both human and non-human, for example: cats, dogs, sheep, cattle, horses and the like (i.e., veterinary use), includes the step of providing a topical composition containing a blend of four separate creams. The blend includes a first cream that contains vitamin A, vitamin D and a base. The base can be petrolatum or lanolin. The blend further includes a second cream that contains povidone-iodine (polyvinylpyrrolidone/iodine) and an emollient base, and a third cream that contains ichthammol and an emollient base. Finally, the blend also includes a fourth cream that contains oil of thuja, zinc oxide, scarlet oil and petrolatum base. The zinc oxide is always employed in conjunction with oil of thuja. The harmful side-effects of zinc oxide can be minimized by employing “thuja zinc”. Preferably, the blend of the four topical creams contains about four pounds of the first cream, about one pound of the second cream, about one pound of the third cream and about one pound of the fourth cream. It is within the scope of the present invention to employ fractions of the above-disclosed weights, or multiples of the above-disclosed weights; provided the ratios of the four topical creams remain constant.

In addition to the blend of four creams, the topical composition includes a steroidal material that contains at least one steroid and a base of white petrolatum; an injectable composition that contains vitamin A, vitamin D and a liquid carrier; and an antibiotic material that contains at least one antibiotic. Optional ingredients include aloe vera, a topical anaesthetic such as lidocaine, an antifungal agent such as miconazole and an anti-parasitic agent. It is within the scope of the present invention to employ a mixture of optional ingredients, such as a mixture of aloe vera and lidocaine.

Another optional ingredient that can be employed under controlled circumstances is dimethylsulfoxide (DMSO). DMSO is a potent anti-inflammatory. It also dissolves almost any organic compound; and therefore it can absorb almost any ingredient into the skin, including unwanted impurities.

Preferably, the steroidal material is present in the topical composition in an amount of about one pound. It is within the scope of the present invention to employ fractions of the above-disclosed weight, or multiples of the above-disclosed weight; provided the ratios of the steroidal material and the four topical creams remain constant. Preferably, the injectable composition is present in the topical composition in an amount of about 30 ml to about 50 ml. It is within the scope of the present invention to employ fractions of the above-disclosed volumes, or multiples of the above-disclosed volumes; provided the ratios of the injectable composition, the steroidal material and the four topical creams remain constant. Preferably, the antibiotic material is present in the topical composition in an amount of about 30 ml to about 50 ml. It is within the scope of the present invention to employ fractions of the above-disclosed volumes, or multiples of the above-disclosed volumes; provided the ratios of the antibiotic material, the injectable composition, the steroidal material and the four topical creams remain constant. If present, the optional ingredient(s) is contained in the topical composition in an amount of about 30 ml to about 50 ml. It is within the scope of the present invention to employ fractions of the above-disclosed volumes, or multiples of the above-disclosed volumes; provided the ratios of the optional ingredient(s), the antibiotic material, the injectable composition, the steroidal material and the four topical creams remain constant.

The method further includes applying a pharmaceutically effective amount of the topical composition to the site of the condition. By pharmaceutically effective amount is meant an amount that is necessary to accelerate the healing process. It is within the scope of the present invention to apply more than one coat of the topical composition to the site of the skin condition. Application of the topical composition can result in a thin film of composition over the site of the condition, or a thicker film. Preferably, the coating of the topical composition on the site of the skin condition has a thickness of about 1/32 inches to about ¼ inches.

The method further involves wrapping the site of the condition, allowing the condition to heal and unwrapping a healed site of the condition. Preferably, the site is wrapped with a sterile bandaging device. The bandaging device can be an elastomeric sleeve, a gauze material, a bandage, a wrapping cloth or the like. In an embodiment, the condition is allowed to heal for a selected time period of about three days to about seven days. After an acceptable time period, the sterile bandaging device is removed. If the site of the condition is not sufficiently healed after the time period, it is within the scope of the present invention to re-apply the topical composition and re-wrap the site with a sterile bandaging device. In another embodiment of the present invention, the site of the condition is coated with the topical composition and wrapped with the sterile bandaging device on a pre-selected schedule, for example, every 24 hours. If advised by competent professionals, the site of the condition can be cleaned with an antibacterial soap or the like after every removal of the bandaging device and prior to application of a fresh coat of the topical composition.

Steroids useful in the present method are hydrocortisone, betamethasone, dexamethasone, flumethasone, isoflupredone, methylprednisolone, prednisone, prednisolone, triamcinalone, fludricortisone, paramethasone and clobetasol. Pharmaceutically acceptable derivatives of each of the above-listed steroids can also be employed in the present method of the invention. It is within the scope of the present invention to employ a mixture of steroids.

Antibiotics useful in the method of the present invention are cephalexin, cephadrine, cefaclor, cefuroxime, cefodroxil, cefpodoxime, cefixime, orbifloxacin, enrofloxacin, ciprofloxacin, difloxacin, marbofloxacin, danofloxacin, amikacin, kanamycin, gentamycin, neomycin, polymyxin, bacitracin, trimethoprim sulfa, sulfadimethoxine, azithromycin, erythromycin, clarithromycin, clindamycin, tetracycline, oxytetracycline, chlortetracycline, doxycycline, florfenicol and spectinomycin. Pharmaceutically acceptable derivatives of each of the above-listed antibiotics can also be employed in the present method of the invention. It is within the scope of the present invention to employ a mixture of antibiotics.

The topical composition of the present invention has a creamy smooth, easily spreadable texture. After the topical composition has been prepared, it is stored in suitable containers that are air-tight and light resistant. The containers are preferably stored in a controlled environment at a temperature of about 20-30 degrees C. The containers remain tightly closed when not in use. The topical composition is intended for veterinary use. It can also be employed in the area of human dermatology or the like. The composition is not intended for ophthalmic use. The composition exhibits the properties of a protective, an emollient, an astringent, a disinfectant, an anti-bacterial, an anti-fungal, an anti-inflammatory, an anti-parasitic and a counter-irritant.

A preferred formulation for the topical composition contains a mixture of the following ingredients: four pounds of a first cream, one pound of a second cream, one pound of a third cream and one pound of a fourth cream. The first cream is a mixture of vitamins E, A and D, and an emollient base. The second cream is povidone-iodine (10%) cream. The third cream is ichthammol(20%) cream. The fourth cream is thuja-zinc oxide ointment. The preferred formulation further contains one pound of triamcinolone acetonide(0.1%) cream as the steroidal material; 33 ml of an injectable composition containing vitamins A and D; and 25 ml of Nuflor™ as the antibiotic material. It has been observed that the preferred formulation for the topical composition of the present invention exhibits the quality of adhering tenaciously to wounds, particularly wet wounds. This specific formulation described above is hereinafter referred to as TOPICAL A.

### EXAMPLES

A 65-yr. old man, employed as a maintenance engineer at a saw mill, experienced chronic discomfort and pain. The man had dry, cracked skin on his hands. His hands were very rough, due to constant exposure to chemicals such as organic solvents, lubricants, grease and degreasers. The various over-the-counter creams and ointments employed in the past were ineffective in treating the skin condition. Medications which were prescribed by various dermatologists were also employed. Again, there was no significant success in the treatment of the dry, cracked skin. After the patient was treated with TOPICAL A, significant healing was observed in three days. The skin of both hands became pain free and relatively smooth, with only minor amounts of cracked skin.

A 55-yr. old woman, employed as a receptionist, experienced chronic pruritic dermatitis on her legs. Lesions, crusts and scabs were the result of the condition. Scratching by the patient contributed to secondary lesions on the legs. The woman had been treated by both a general practice physician and a specialist in dermatology. She had been on a regimen of antibiotics, steroids and antihistamines. The patient was treated by applying TOPICAL A to both legs, followed by wrapping the legs with sterile bandages. After 48 hours, the sterile bandages were removed. The lesions were substantially healed, although some discoloring remained.

A 52-yr. old woman, employed as a veterinary technician, suffered a woodstove burn on her right forearm. After 2½ weeks, the burned area remained red, cracked and crusty. The area also bled very easily. TOPICAL A was liberally applied to the affected area. The treated area was wrapped with a sterile bandage. The sterile bandage was removed after 48 hours. The burned area was substantially healed. Re-epithelialization was observed.

A 5 yr. old beagle was hit by a car. Third and fourth digits of a distal paw were beyond repair. The remaining toes suffered partial degloving injury. The third and fourth digits were amputated. The remaining skin was sutured over the defect, covered with TOPICAL A and wrapped with a sterile bandage. After seven days, the sterile bandage was removed, and there appeared to be some ischemic injury to the suture line. The area was debrided, re-sutured and stapled. The area was re-covered with TOPICAL A, and re-wrapped with a sterile bandage. After another seven days, the sterile bandage, sutures and staples were removed. The suture line appeared to be at least 90% healed.

A 2 yr. old cat was hit by a car. The cat suffered degloving injuries to the hock and the metatarsal region. The cat further suffered a compound fracture of a distal tibia. Both the bone end and soft tissue area were contaminated. Because of a lack of funds, orthopaedic surgery was not an option. Therefore, surgery was conducted to debride and repair the degloving injuries. The repaired area was coated with TOPICAL A and then wrapped with a sterile bandage. A splint was then applied. After 10 days, the splint was was checked. Observation of the surgically repaired area revealed almost complete healing of the soft tissue. Although some granulation tissue remained, most of the wound re-epithelialized.

A pack of field trial fox hounds, 12 in total, is treated with TOPICAL A by rubbing the formulation on the foot pads of the fox hounds. The hounds are employed in competitive field trials for an entire season. Treatment with TOPICAL A is performed after each competition, and at least once between competitions. The fox hounds respond by recovering more quickly than in previous seasons. Also, the hounds experience reduced incidences of lameness during the season.

While not wishing to be bound by theory, we believe that the various ingredients in the TOPICAL A composition, as well as other compositions of the present invention, act in synergistic fashion to accelerate the healing process. The healing process is accelerated by effectively stimulating the growth of new epithelial cells. It appears that the amount of scar tissue is reduced compared to use of prior art compositions, including both over-the-counter and prescription compositions. Preferably, the composition of the present invention is applied liberally to the wounded skin and then wrapped to insure continuous contact between the composition and the wounded skin.

The TOPICAL A composition and related compositions of the present invention have various properties such as anti-bacterial, anti-fungal, anti-inflammatory, anti-parasitic, emollient and counter-irritant. The various ingredients in the TOPICAL A composition and related compositions of the present invention work in a synergistic fashion to promote and accelerate wound healing.

While the invention has been described by specific examples and embodiments, there is no intent to limit the inventive concept except as set forth in the following claims.

I claim:

1. A topical composition for the treatment of mammals suffering from a condition of the skin such as a skin lesion, a wound, a cut, an abrasion, a hot spot, a sore foot pad, inflamed skin, infected skin and dry skin, the composition comprising:
(a) a blend of a first cream, a second cream, a third cream and a fourth cream, wherein the first cream comprises vitamin A, vitamin D and a base selected from the group consisting of petrolatum and lanolin; the second cream comprises povidone-iodine (polyvinylpyrrolidone/iodine) and an emollient base; the third cream comprises ichthammol and an emollient base; and the fourth cream comprises oil of thuja, zinc oxide, scarlet oil and petrolatum base;
(b) a steroidal material comprising at least one steroid and an emollient base;
(c) an injectable composition comprising vitamin A, vitamin D and a liquid carrier;
(d) an antibiotic material comprising at least one antibiotic; and
(e) optionally, an ingredient selected from the group consisting of aloe vera, a topical anaesthetic, DMSO, an anti-parasitic agent, an antifungal agent and mixtures thereof.

2. The composition of claim 1 wherein the at least one steroid is a member selected from the group consisting of hydrocortisone, betamethasone, dexamethasone, flumethasone, isoflupredone, methylprednisolone, prednisone, prednisolone, triamcinalone, fludricortisone, paramethasone and clobetasol.

3. The composition of claim 1 wherein the at least one antibiotic is a member selected from the group consisting of cephalexin, cephadrine, cefaclor, cefuroxime, cefodroxil, cefpodoxime, cefixime, orbifloxacin, enrofloxacin, ciprofloxacin, difloxacin, marbofloxacin, danofloxacin, amikacin, kanamycin, gentamycin, neomycin, polymyxin, bacitracin, trimethoprim sulfa, sulfadimethoxine, azithromycin, erythromycin, clarithromycin, clindamycin, tetracycline, oxytetracycline, chlortetracycline, doxycycline, florfenicol and spectinomycin.

4. The composition of claim 1 wherein the blend of the four topical creams contains about four pounds of the first cream, about one pound of the second cream, about one pound of the third cream, and about one pound of the fourth cream.

5. The composition of claim 4 wherein the steroidal material is present in an amount of about one pound; the injectable composition is present in an amount of about 30 ml to about 50 ml; the antibiotic material is present in an amount of about 30 ml to about 50 ml; and the optional ingredient is present in an amount of about 30 ml to about 50 ml.

6. A method for preparing a topical composition for the treatment of mammals suffering from a condition of the skin such as a skin lesion, a wound, a cut, an abrasion, a hot spot, a sore foot pad, inflamed skin and dry skin, the method comprising:
(a) providing a mixing device;
(b) heating the mixing device to a temperature of about 30 degrees C. to about 50 degrees C.;
(c) adding to the heated mixing device a first cream comprising vitamin A, vitamin D and a base selected from the group consisting of petrolatum and lanolin;
(d) adding to the heated mixing device a second cream comprising povidone-iodine (polyvinylpyrrolidone/iodine) and an emollient base;
(e) optionally, activating the mixing device to blend the first cream and the second cream;
(f) adding to the heated mixing device a third cream comprising ichthammol and an emollient base;
(g) optionally, activating the mixing device to blend the first, second and third creams;
(h) adding to the heated mixing device a fourth cream comprising oil of thuja, zinc oxide, scarlet oil and petrolatum base;
(i) optionally, activating the mixing device to blend the first, second, third and fourth creams;
(j) adding to the heated mixing device a steroidal material comprising at least one steroid and an emollient base;
(k) optionally, activating the mixing device to blend the first, second, third, and fourth creams, and the steroidal material;
(l) adding to the heated mixing device an injectable composition comprising vitamin A, vitamin D and a liquid carrier;
(m) optionally, activating the mixing device to blend the first, second, third and fourth creams, the steroidal material, and the injectable composition;
(n) adding to the heated mixing device an antibiotic material comprising at least one antibiotic;
(o) optionally, activating the mixing device to blend the first, second, third and fourth creams, the steroidal material, the injectable composition and the antibiotic material;
(p) optionally, adding to the heated mixing device an ingredient selected from the group consisting of aloe vera, a topical anaesthetic, an antifungal agent, DMSO, an anti-parasitic agent and mixtures thereof;
(q) activating the mixing device to blend the first, second, third and fourth creams, the steroidal material, the injectable composition, the antibiotic material and the optional ingredient(s) to obtain the topical composition; and
(r) withdrawing the topical composition from the mixing device.

7. The method of claim 6 wherein the at least one steroid is a member selected from the group consisting of hydrocortisone, betamethasone, dexamethasone, flumethasone, isoflupredone, methylprednisolone, prednisone, prednisolone, triamcinalone, fludricortisone, paramethasone and clobetasol.

8. The method of claim 6 wherein the at least one antibiotic is a member selected from the group consisting of cephalexin, cephadrine, cefaclor, cefuroxime, cefodroxil, cefpodoxime, cefixime, orbifloxacin, enrofloxacin, ciprofloxacin, difloxacin, marbofloxacin, danofloxacin, amikacin, kanamycin, gentamycin, neomycin, polymyxin, bacitracin, trimethoprim sulfa, sulfadimethoxine, azithromycin, erythromycin, clarithromycin, clindamycin, tetracycline, oxytetracycline, chlortetracycline, doxycycline, florfenicol and spectinomycin.

9. The method of claim 6 wherein the first cream is present in an amount of about four pounds; the second cream is present in an amount of about one pound; the third cream is present in an amount of about one pound; and the fourth cream is present in an amount of about one pound.

10. The method of claim 9 wherein the steroidal material is present in an amount of about one pound; the injectable composition is present in an amount of about 30 ml to about 50 ml; the antibiotic material is present in an amount of about 30 ml to about 50 ml; and the optional ingredient is present in an amount of about 30 ml to about 50 ml.

11. A method for the treatment of mammals suffering from a condition of the skin such as a skin lesion, a wound, a cut, an abrasion, a hot spot, a sore foot pad, inflamed skin and dry skin, the method comprising:
(a) providing a topical composition comprising: a blend of four creams; a steroidal material comprising at least one steroid; an injectable composition comprising vitamin A and vitamin D; an antibiotic material comprising at least one antibiotic; and, optionally, an ingredient selected from the group consisting of aloe vera, a topical anaesthetic, an anti-fungal agent, an anti-parasitic agent, DMSO and mixtures thereof; wherein a first cream comprises vitamin A, vitamin D and a base selected from the group consisting of petrolatum and lanolin, a second cream comprises povidone-iodine (polyvinylpyrrolidone/iodine), a third cream comprises ichthammol and an emollient base, and a fourth cream comprises oil of thuja, zinc oxide, scarlet oil and petrolatum base
(b) applying a pharmaceutically effective amount of the topical composition over a site of the condition;
(c) wrapping the site of the condition with a sterile bandaging device;
(d) allowing the condition to heal for a time of about three days to about seven days; and
(e) removing the sterile bandaging device.

12. The method of claim 11 wherein the at least one steroid is a member selected from the group consisting of hydrocortisone, betamethasone, dexamethasone, flumethasone, isoflupredone, methylprednisolone, prednisone, prednisolone, triamcinalone, fludricortisone, paramethasone and clobetasol.

13. The method of claim 11 wherein the at least one antibiotic is a member selected from the group consisting of cephalexin, cephadrine, cefaclor, cefuroxime, cefodroxil, cefpodoxime, cefixime, orbifloxacin, enrofloxacin, ciprofloxacin, difloxacin, marbofloxacin, danofloxacin, amikacin, kanamycin, gentamycin, neomycin, polymyxin, bacitracin, trimethoprim sulfa, sulfadimethoxine, azithromycin, erythromycin, clarithromycin, clindamycin, tetracycline, oxytetracycline, chlortetracycline, doxycycline, florfenicol and spectinomycin.

14. The method of claim 11 wherein the blend of four creams consists essentially of about four pounds of the first cream, about one pound of the second cream, about one pound of the third cream and about one pound of the fourth cream.

15. The method of claim 14 wherein the steroidal material is present in an amount of about one pound; the injectable composition is present in an amount of about 30 ml to about 50 ml; the antibiotic material is present in an amount of about 30 ml to about 50 ml; and the optional ingredient is present in an amount of about 30 ml to about 50 ml.
